(19) United States
(12) Patent Application Publication
(10) Pub. No.: US 2016/0303205 A1
(43) Pub. Date: Oct. 20, 2016

(54) Combination Therapies for Lysosomal Storage Diseases

(71) Applicant: Washington University, Saint Louis, MO (US)

(72) Inventor: Mark Sands, Saint Louis, MO (US)

(21) Appl. No.: 15/097,665

(22) Filed: Apr. 13, 2016

Related U.S. Application Data
(60) Provisional application No. 62/148,573, filed on Apr. 16, 2015.

(51) Int. Cl. A61K 38/47; A61K 35/28; A61K 45/06; A61K 31/42

(52) U.S. Cl. CPC A61K 38/47; A61K 31/42; A61K 35/28; A61K 45/06; C12Y 302/01046

(57) ABSTRACT

Disclosed are methods of treating lysosomal storage diseases, including Globoid Cell Leukodystrophy (GSD). GSD is refractory to standard treatments, where even more invasive treatments only provide minor benefits. However, combinations of three treatments can interact synergistically to provide marked extension of life-span and increases in neuronal function. A combination of a primary treatment such as a gene therapy or an enzyme replacement therapy and at least two secondary therapies such as a substrate reduction therapy and an immunomodulation treatment can lead to increased average life expectancy compared to any individual treatment or pair of treatments.

### CROSS REFERENCE TO RELATED APPLICATION

This application claims the benefit of and priority to U.S. Provisional Application 62/148,573, filed Apr. 16, 2015, which is hereby incorporated by reference in its entirety.

### STATEMENT OF GOVERNMENT INTEREST

This invention was made with government support under R01 NS084861 awarded by the National Institutes of Health. The government has certain rights in the invention.

### INTRODUCTION

There are over 50 known lysosomal storage diseases (LSDs), each involving a different genetic defect. One LSD, Globoid Cell Leukodystrophy (GLD, Krabbe Disease), is an inborn error of metabolism resulting from a deficiency in the lysosomal enzyme galactocerebrosidase (GALC) (Wenger et al., The Metabolic & Molecular Basis of Disease 8 Edition pp 2669-3694, 2001). In the absence of GALC activity, the cytotoxic lipid galactosylsphingosine (psychosine) accumulates in the central (CNS) and peripheral (PNS) nervous systems. Loss of GALC activity results in the accumulation of a cytotoxic lipid, galactosylsphingosine (psychosine), in the central (CNS) and peripheral (PNS) nervous systems, and leads to a dysmyelinating phenotype (Miyatake and Suzuki, Birth Defects Orig Artic Ser 9:136-140, 1973). Globoid Cell Leukodystrophy has proven refractory to most therapeutic interventions. This is likely due to the complex nature of this disease, involving the primary and multiple secondary pathogenic mechanisms.

The currently available treatment for GLI) is hematopoietic stem cell transplantation (HSCT), which provides only modest improvements if initiated prior to symptom onset (Martin et al., Biol. Blood Marrow Transplant. 12: 184-194, 2006; Krivit et al. New Eng. J. Med. 338: 1119-1126, 1998; Escolar et al. New Engl. J. Med. 352:2069-2081, 2005; Sakai, N., Brain Dev. 31: 485-487, 2009). Although hematopoietic stem cell transplantation (HSCT) therapy provides modest benefit in both pre-symptomatic children and the murine model (Twitcher), there is no cure for GLD. In addition, GLD has been relatively refractory to virtually every experimental therapy attempted.

A mouse model of GLD, the Twitcher mouse, has been available for more than three decades (Kobayashi et al., Brain Res 202:479-483, 1980). This mouse model mimics the human disease, and has a rapid and severe disease course (Suzuki and Suzuki, Am. J. Path. 111:394-341, 1983). Median survival of Twitcher mice is 42 days; range, 30-47 days (Yeager, A. M., et al., Transplantation 56: 185-190, 1993). Despite the availability of the Twitcher mouse, development of effective therapies has been slow. Virtually all single therapy approaches, including bone marrow transplant (BMT) (Yeager et al, Science 225:1052-1054, 1984), gene therapy (Lin et al, Molecular Therapy 12:422-430, 2005; Lin et al., Molecular Therapy 15:44-52, 2007; Galbiati et al., J. Neurosci. Rev. 87:1748-1759, 209; Gentner et al, 2010 Sci. Transl. Med. 2:58ra84; Reddy et al., J. Neurosci. 31:9945-9957, 2011; Rafi et al, Molecular Therapy 20:2031-2042, 2012), substrate reduction (LeVine et al., J. Neurosci. Res. 60:231-236, 2000; Biswas and LeVine, Pediatr. Res. 51:40-47, 2002), antioxidants (lHawkins-Salsbury, J. A., et al., Exp. Neurol. 237: 444-452, 2012), steroids (Kagitani-Shimono et al., Journal of Neuroinflammation 2:10 2005; Luzi et al., Brain Res. 1300:146-158, 2009), enzyme replacement therapy (Lee et al., FASEB J 19:1549-1551, 2005; Qin et al., Mol. Genet. Metab. 107:186-196, 2012), and neuronal (Pellegatta et al., Neurobiol. Dis. 21:314-323, 2006) or mesenchymal (Ripoll et al., Stem Cells 29:67-77, 2011) progenitor cell transplantation, have met with minimal to modest success.

Combination therapies that target both the primary mechanism and a secondary consequence have been more successful in treating globoid cell leukodystrophy. Intracranial gene therapy targeting the primary genetic defect synergized with BMT to nearly triple the litfespan of the Twitcher mouse (Lin et al., Molecular Therapy 15:44-52, 2007). Subsequent improvements in the route of gene therapy delivery further extended the life span in the Twitcher mouse model (Reddy et al., J. Neurosci. 31:9945-9957, 2011).

Substrate reduction therapy (SRT) for LSDs decreases the rate of accumulation of undegraded metabolites by reducing the synthesis of the enzyme substrates. This can be accomplished in GLD with administration of L-cycloserine, which decreases the synthesis of sphingolipids, including psychosine (Sundaram and Lev, J Lipid Res 26:473-477, 1985; LeVine, S. M., et al., 2000). L-cycloserine administration alone improves lifespan in the Twitcher mouse to ˜57 days (LeVine et al., J Neurosci Res. 60: 231-236, 2000), and when combined with BMT, increases lifespan to ˜112 days (Biswas and LeVine, Pediatr Res 51:40-47, 2002).

### SUMMARY

The present inventors have developed methods of treating lysosomal storage diseases such as, without limitation, globoid-cell leukodystrophy (GLD). In various embodiments, the methods combine multiple therapeutic approaches to increase synergistically the efficacy of various individual lysosomal storage disease treatments. In various embodiments, the methods can provide for increased lifespan and/or quality of life to an extent greater than that expected for any one form of treatment by itself or in combination with one other form of therapy, i.e., any pairings of two treatments. In various embodiments, the methods combine three or more treatments, such as a treatment that targets the primary pathogenic mechanism and at least two other treatments that target secondary consequences of an LSD such as globoid-cell leukodystrophy (GLD).

In some embodiments, the present teachings include methods of treating a subject having a lysosomal storage disease. In various configurations, these methods can comprise combinations of three or more treatments, such as a combination comprising a primary treatment such as a gene therapy or an enzyme replacement therapy, and at least two secondary treatments such as a substrate reduction therapy and an immunomodulation therapy. In some embodiments, the lysosomal storage disease can be globoid cell leukodystrophy.

In some configurations, a gene therapy can comprise, consist essentially of, or consist of administration of a therapeutically effective amount of a vector such as a virus which harbors a nucleic acid that encodes an enzyme deficient in a lysosomal storage disease, such as galactocerebrosidase (GALC). In some configurations, a viral vector can be an adeno-associated virus (AAV), an adenovirus, a lentivirus, a herpes virus, or a retrovirus such as a gamma-retroviral vector. In various configurations, an AAV virus can be an AAV25, an AAVrh10, an AAV2/9, or an AAV vector genome pseudotyped with any number of naturally occurring or engineered capsid proteins. In some configurations, a vector such as an AAV virus can comprise a CMV enhancer. In some configurations, a vector such as an AAV virus can comprise a chicken O-actin promoter. In some configurations, a vector such as an AAV virus can comprise a CMV enhancer and a chicken f-actin promoter. In some configurations, a vector such as an AAV virus can comprise a CMV enhancer and a chicken 3-actin promoter, each of which can be operably linked to a nucleic acid that encodes an enzyme deficient in a lysosomal storage disease, such as galactocerebrosidase (GALC).

In some configurations, an enzyme replacement therapy can comprise, consist essentially of, or consist of administration of a deficient enzyme, such as galactocerebrosidase. In some aspects, the administration of the deficient enzyme can comprise, consist essentially of, or consist of intrathecal or intracerebroventricular injection of galactocerebrosidase in a therapeutically effective amount, such as, without limitation, weekly injections or chronic administration through an implanted pump/port configuration. Administration of the deficient enzyme can also comprise, consist essentially of, or consist of intravenous administration of a recombinant lysosomal enzyme engineered to cross the blood brain barrier. In some configurations, the galactocerebrosidase can be a recombinant galactocerebrosidase. In some configurations, the galactocerebrosidase can be a human galactocerebrosidase, such as a recombinant human galactocerebrosidase.

In some configurations, a substrate reduction therapy can comprise, consist essentially of, or consist of administration of an inhibitor of an enzyme that acts immediately upstream of galactosylceramide and galactosylsphingosine (psychosine) such as ceramide galactosyltransferase (CGT).

In some configurations, a substrate reduction therapy can comprise, consist essentially of, or consist of administration of a therapeutically effective amount of an inhibitor of an enzyme further upstream that participates in ceramide synthesis such as 3-ketodyhydrosphingosine synthase. In some configurations, substrate reduction therapy can comprise, consist essentially of, or consist of administration of a therapeutically effective amount of an inhibitor of 3-ketodyhydrosphingosine synthase such as, for example, L-cycloserine.

In some configurations, an immunomodulation can comprise, consist essentially of, or consist of bone marrow transplantation and/or administration of a therapeutically effective amount of a JAK-STAT inhibitor such as, without limitation,

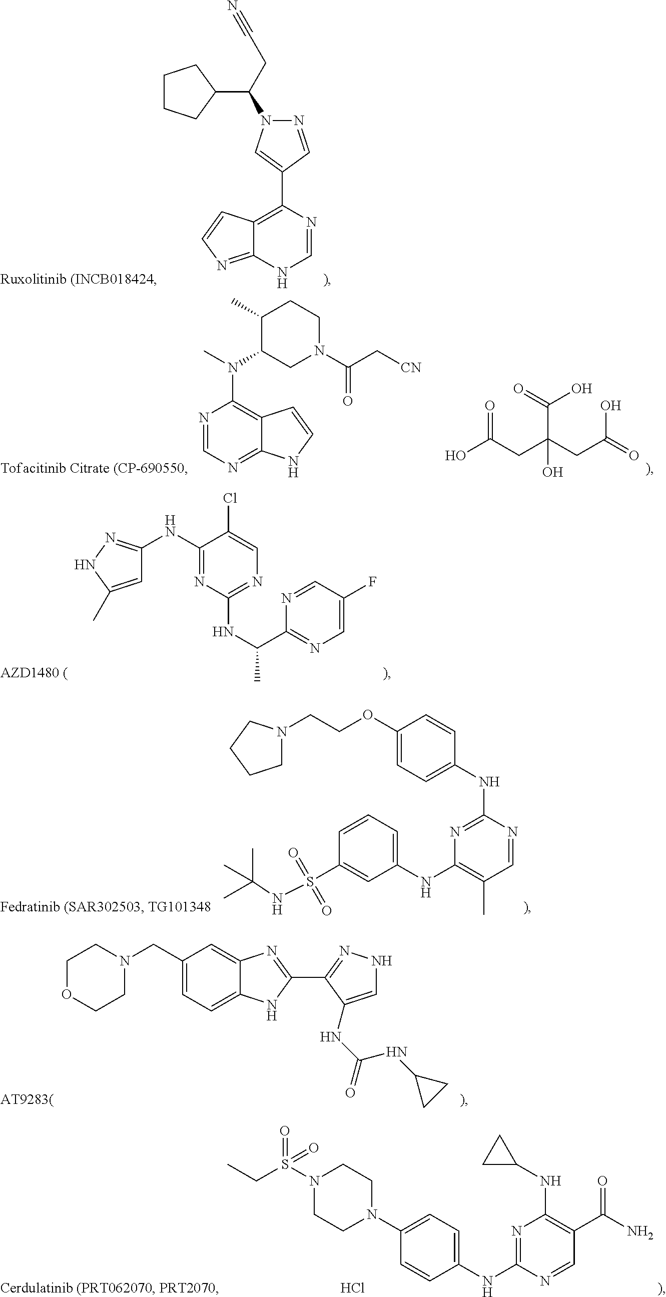

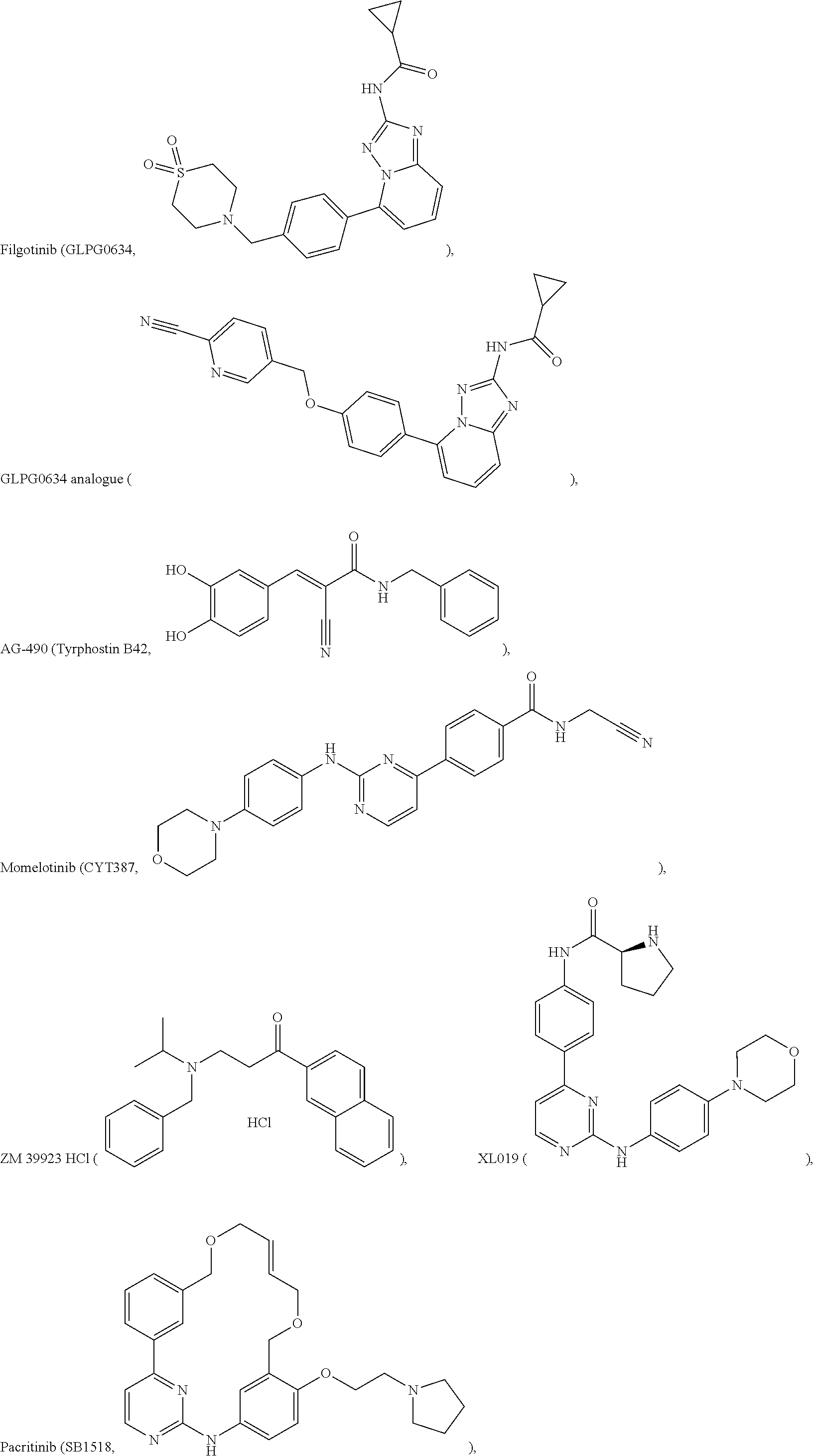

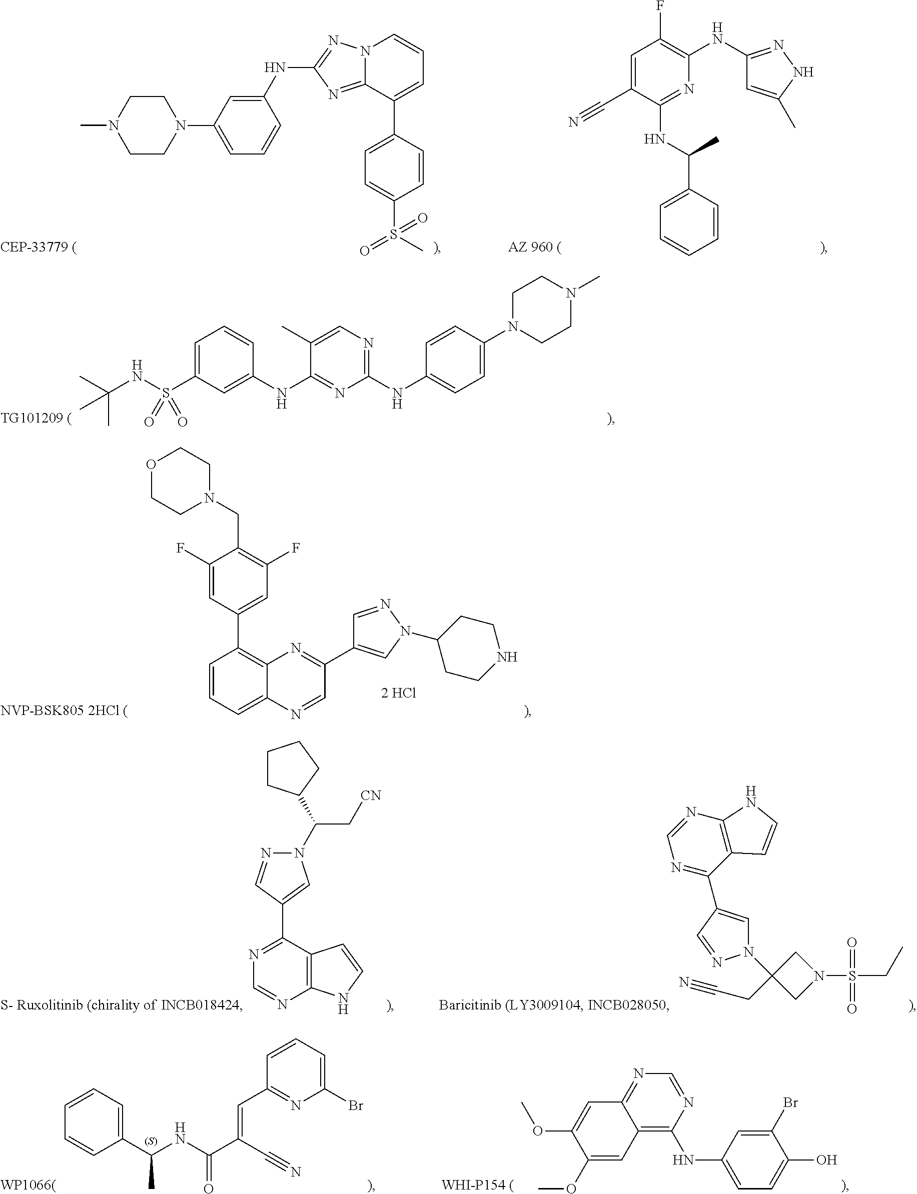

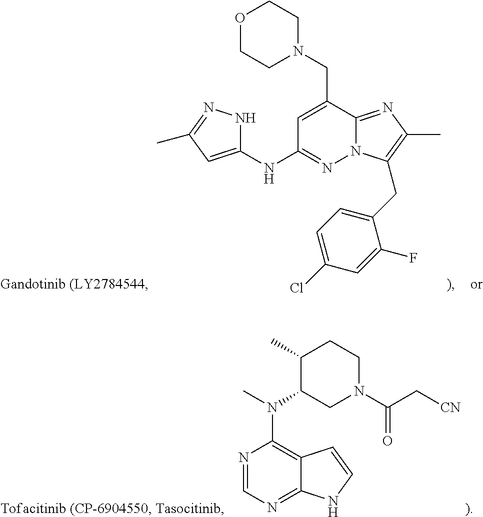

In some configurations, the subject can be a mammal. In some configurations, the mammal can be a human, such as a human in need of treatment for a lysosomal storage disease such as, without limitation, globoid-cell leukodystrophy (GLD).

In some embodiments, the present teachings include a combination of more than three therapies in the treatment of a lysosomal storage disease such as, without limitation, globoid-cell leukodystrophy (GLD), wherein at least three therapies comprise a primary therapy such as gene therapy or enzyme replacement therapy, a substrate reduction therapy and an immunomodulation therapy. In some configurations, an additional therapy can comprise administration of one or more antioxidants and/or vitamins that lower homocysteine levels, such as, for example, N-acetylcysteine, vitamin C, vitamin E, vitamin B6, vitamin B112 and/or folic acid. In some configurations, an additional therapy can comprise administration of one or more therapeutic agents that prevent axonal damage or promote restoration of damaged axons, such as, without limitation, a microtubule stabilizing compound such as paclitaxel (laxol).

### BRIEF DESCRIPTION OF THE DRAWINGS

The patent or application file contains at least one drawing executed in color. Copies of this patent or patent application publication with color drawing(s) will be provided by the Office upon request and payment of the necessary tiee.

FIG. 1 illustrates a three-prong strategy of the present teachings for treatment of Globoid Cell Leukodystropy, comprising targeting GALC deficiency by gene therapy, substrate reduction therapy (to inhibit psychosine synthesis), and bone marrow transplantation (for treating neuroinflammation).

FIG. 2A-D illustrates that a triple therapy of the present teachings can (A) increase lifespan and (13) increase body weight, and can enhance motor function in Twitcher mice in (C) Rotarod and (D) Wirehang behavioral assays.

FIG. 3A-D illustrates that a triple therapy of the present teachings can (A) supply persistent GALC activity and (B, C, D) decrease psychosine accumulation in Twitcher mice.

FIG. 4 illustrates that persistent donor chimerism can be observed in the bone marrow and brains of Twitcher mice subjected to a triple-treatment of the present teachings.

FIG. 5 illustrates that myelin can be well preserved in Twitcher mice subjected to a triple-treatment of the present teachings compared to control Twitcher mice receiving 0, 1 or 2 treatments.

FIG. 6A-B illustrate that microglial and astrocyte activation can be reduced by a triple therapy of the present teachings.

FIG. 7 illustrates that a triple therapy of the present teachings can reduce cytokineichemokine expression in the Twitcher brain.

### DETAILED DESCRIPTION

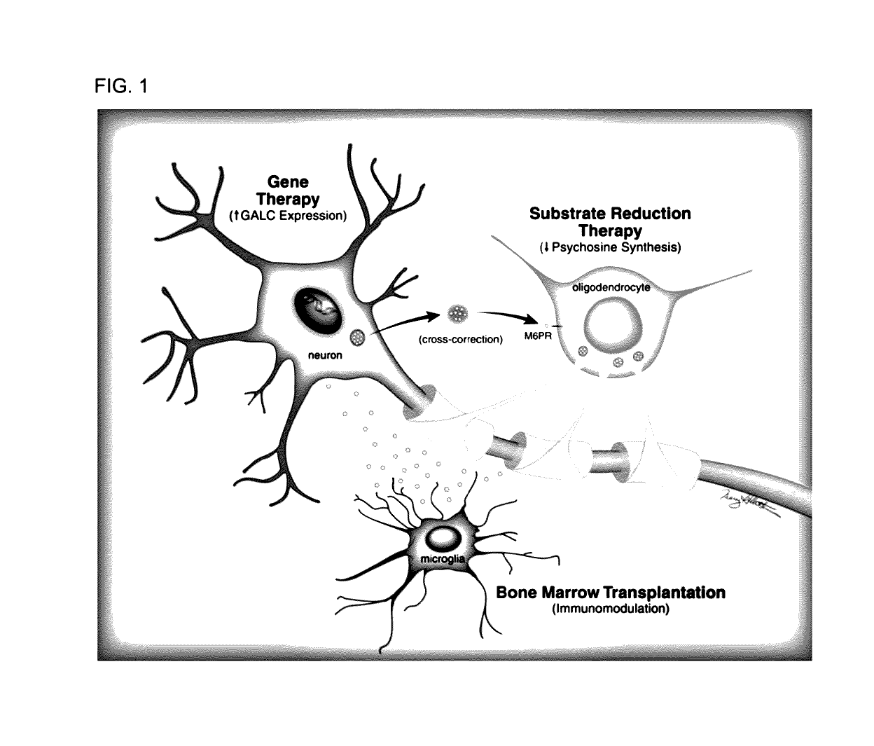

In the current study, the inventors targeted the primary defect in Twitcher mice (i.e., a mutation in a gene encoding galactocerebrosidase) with AAV-mediated, CNS-directed gene therapy (FIG. 1). Without being limited by theory, neurons can be transduced and can express GALC following intracranial injection of an AAV2/5 gene transfer vector. Two secondary consequences of GALC deficiency, psychosine accumulation and neuroinflammation, can be targeted with substrate reduction therapy and BMT, respectively (FIG. 1). Without being limited by theory, secreted GALC can bind the mannose-6-phosphate receptor (M6PR) on non-transduced cells and can be endocytosed. Without being limited by theory, endocytosed GALC can be targeted to the lysosomes of oligodendrocytes where it can degrade psychosine. Without being limited by theory, a substrate reduction therapy drug, which can be in some embodiments an inhibitor of 3-ketodyhydrosphingosine synthase such as L-cycloserine. Without being limited by theory, an inhibitor of 3-ketodyhydrosphingosine synthase can act on oligodendrocytes to decrease the synthesis of psychosine and thereby slow the accumulation of psychosine. Without being limited by theory, bone marrow transplantation can serve at least two functions in globoid cell leukodystrophy: 1) it can decrease either or both of a disease- and a viral vector-associated neurinflammatory response, and 2) it can secrete GALC enzyme as donor-derived microglia reconstitute the CNS. This multi-pronged approach resulted in the greatest increase in lifespan to date, with triple-treated Twitcher mice living to a median of ˜300 days and several mice living >450 days. Based on the increases in life span when applied singly, the combination of these three treatments should result in a median lifespan of ˜90 days if they interacted in an additive fashion. The unprecedented degree of synergy indicates that combining therapies directed at multiple targets can provide health benefits in treatments of complex metabolic disorders such as Globoid Cell Ieukodystrophy.

The present teachings include methods of triple treatment of lysosomal storage diseases. These methods can include treatment of a subject with multiple therapeutic approaches to combine enhanced results. Potential therapeutic approaches can include gene therapy, BMT, substrate reduction, antioxidants, steroids, enzyme replacement or progenitor cells. Combining gene therapy, BMT and substrate reduction therapies can produce synergistic results that are greater than any therapy alone or a combination of two therapies, such as a combination of substrate reduction and BMT without gene therapy. In some configurations, gene therapy can include AAV mediated gene therapy. In some configurations, substrate reduction therapy can include an inhibitor of 3-ketodyhydrosphingosine synthase such as, without limitation, L-cycloserine.

In the present teachings, Twitcher mice were simultaneously treated with CNS-directed gene therapy, substrate reduction therapy, and bone marrow transplantation. Without being limited by theory, these three treatments target the primary pathogenic mechanism, i.e., GALC deficiency, and two secondary consequences of GALC deficiency, i.e., psychosine accumulation and neuroinflammation. In various configurations, simultaneously treating multiple pathogenic targets by can result in an increase in life span, and can further include enhanced motor function, persistent GALC expression, nearly normal psychosine levels, and decreased neuroinflammation.

Methods and compositions described herein utilize laboratory techniques well known to skilled artisans, and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; Nagy, A., Manipulating the Mouse Embryo: A Laboratory Manual (Third Edition), Cold Spring Harbor, N.Y., 2003 and Harlow, E., Using Antibodies: A Laboratory Manual, Cold Spring Harbor Laboratory Press, Cold Spring Harbor, N.Y., 1999. Methods of administration of pharmaceuticals and dosage regimes, can be determined according to standard principles of pharmacology well known skilled artisans, using methods provided by standard reference texts such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003. As used in the present description and any appended claims, the singular forms “a”, “an” and “the” are intended to include the plural forms as well, unless the context indicates otherwise.

### Abbreviations

BMT bone marrow transplant

### CNS Central Nervous System

GALC galactocerebrosidase

### GLD Globoid Cell Leukodystrophy (Krabbe Disease)

HSCI hematopoietic stem cell transplantation
LSD lysosomal storage disease

### PNS Peripheral Nervous System

SRT substrate reduction therapy

The present teachings include descriptions that are not intended to limit the scope of any aspect or claim. Unless specifically presented in the past tense, an example can be a prophetic or an actual example. The examples and methods are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Methods

Treated and untreated Twitcher mice as well as untreated controls were used in all experiments. Twitcher mice were treated with CNS-directed gene therapy on post-natal day (PND) 2, BMT on PND 3, and L-cycloserine three times a week starting on PNDS. The experimental and control animals were randomly assigned to groups for analysis at pre-determined time points (n=3-6 animals per time point) or for life span and behavioral analyses (n=10-14 animals per group). The number of animals per group was based on previous experience with this model and the specific assays as well as power calculations. Quantitative biochemical analyses (GALC activity, psychosine measurements, cytokine/chemokine levels) were performed in duplicate on tissues from 3-6 animals, depending on the assay. Semi-quantitative histomorphometry was performed on at least 3 representative sections per animal from at least 3 animals per group. The photography settings were kept constant for all images. Analysis of variance (ANOVA) was used for group comparisons with Bonferroni correction for pair-wise comparisons. Student's t-test was used to compare the means of two groups. Significant differences in life span were determined by log-rank test. Differences were deemed significance at a p-value of <0.05.

### Experimental Animals:

Animals were housed at Washington University School of Medicine. The Institutional Animal Care and Use Committee at Washington University School of Medicine approved all animal protocols. Heterozygous Twitcher (GALC +/−) mice on a C57BLi6 background were obtained from the Jackson Laboratory, and heterozygote by heterozygote matings were used to generate the homozygous twitcher (GALC −/−) mice used in this study. Genotypes were determined on postnatal day 1 by PCR as previously described (Sakai et al. 1996 J Neurochem 66:1118-1124; Lin et al., 2005 Molecular Therapy 12:422-430.). Hematopoietic stem cell donors were sex-matched syngenic GALC +/+ mice expressing GFP under control of the CAGGS promoter (Okabe et al. 1997 FASEB Lett 407:313-319). Mice were housed under standard conditions with ad libitum access to food and water and were maintained on a 12/12 light/dark cycle. Only mice that survived to weaning at 28 d were included in this study.

### Viral Vector:

An AAV 2/5 vector was prepared by the Virus Vector Core Facility at the Gene Therapy Center of the University of North Carolina. The viral vector comprised marine GALC cDNA under control of the CMV enhancer and chicken 3-actin promoter. The murine GALC cDNA was followed by the 3′-untranslated region from the rabbit β-globin gene. The single-stranded AAV2 vector was pseudotyped with an AAV5 capsid. Virus was produced using a triple-transfection method in HEK293 cells and was recovered using sequential step and continuous CsCl gradients, followed by dialysis in PBS containing 350 mM NaCl and 5% sorbitol. Viral titers were determined using dot blot hybridization as previously described (Gray et al. 2011 Hum Gene Ther 22:1143-1153). Virus was diluted to a final concentration of 10¹² viral particles per mL in Lactated Ringer's solution and stored at −80° C.

### Intracranial and Intrathecal Injections:

AAV2/5-GALC was administered to the nervous system on postnatal day two to three. Intracranial injections were performed by hand using the same coordinates as previously described (Reddy et al. 2011 J Neurosci 31:9945-9957). Briefly, the mice were immobilized by hypothermia and the injection site was cleaned with 70% EtOH. Each brain hemisphere received three, 2 μL, (10¹² viral particles per mL.) injections, one in the forebrain, one in the thalamus, and one in the cerebellum. Virus was also administered intrathecally at the same time. To prepare the virus for intrathecal injection, 15 μL of virus (10¹² viral particles per mL) was mixed with three μL of trypan blue. To load the syringe a total of 15 μL of this mixture was drawn up. The mouse was restrained and a needle inserted into the spinal column approximately midway along the back. A successful injection was defined as the presence of blue dye in both the cerebellum and sacrum. The animals were warmed and returned to the mothers. A 50 μL Hamilton syringe fitted with a 32-gauge needle was used for all injections.

### Bone Marrow Transplantation:

Animals receiving BMT were exposed to 400 rads of total body irradiation from a ¹³⁷Cs source on the day following gene therapy administration (PND 3-4). Mice were injected intravenously (Sands and Barker, 1999 Lab Anim Sci 49:328-330) with 10⁶ nucleated donor bone marrow cells immediately after irradiation. Following this myelo-reductive conditioning regimen, treated animals had donor chimerism of 5-30% at 36 d as determined by flow cytometry for GFP.

### L-Cycloserine Treatment:

Treatment with sub-cutaneous L-cycloserine began on PND 5-7 and continued three times per week for the life of the animal. L-cycloserine was diluted in PBS fresh each day, then filter sterilized prior to injection. Animals received 25 mg/kg L-cycloserine until PND28, and 50 mg/kg for the rest of their life.

### LFB/PAS Staining and Immunohistochemistry:

Brains were harvested immediately following perfusion and either flash frozen for biochemical analyses or fixed in 4% paraformaldehyde for 24 hours at 4° C. The fixed brains were then moved to 30% sucrose in tris-buffered saline (TBS) for an additional 24-48 hours at 4° C. Following cryoprotection, the brain was embedded in optimal cutting medium (OCT, Sakura Finetek, Torrance, Calif.), frozen on dry ice and cryosectioned. 16 μm sections were stained with luxol fast blue and periodic acid Schiff (LFB/PAS) as previously described (Lin et al, 2005 Molecular Therapy 12:422-430). Additional sections were immunostained with anti-GFAP, anti-CD68, or anti-GFP antibodies according to standard methods. Briefly, free-floating sections were incubated in 1% hydrogen peroxide to quench endogenous peroxidase activity. Sections were then blocked in normal goat serum and incubated in primary antibody. Sections were next incubated in the appropriate biotinylated secondary antibody, followed by incubation in Vectastain Elite ABC reagent (Vector Laboratories). Sections were developed using a DAB peroxidase substrate kit (Vector Laboratories). Antibodies were as follows: rabbit anti-mouse glial fibrillary acidic protein (GFAP) (1:100; Immunostar), rat anti-mouse CD68 (1:1000; AbD Serotec), rabbit polyclonal anti-mouse GFP (1:10,000; Abeam), biotinylated anti-rabbit IgG supplied with the Vectastain kit (1:200; PK-6101 Vector Laboratories), and mouse-adsorbed biotinylated anti-rat IgG (1:200; Vector Laboratories). Stained sections were mounted, dehydrated and cover slipped. Images were captured using an Olympus BX41 microscope and Olympus DP20 camera. All image capture variables were kept identical. At least three sections per animal and three animals per treatment group were analyzed for each brain area studied. Staining was quantified using ImageJ software. The percentage of total image area with staining above a set threshold value was calculated as the area fraction.

### Statistical Analysis:

Statistical significance was calculated using a one-way ANOVA followed by Bonferroni correction for multiple comparisons in order to compare all treatment groups unless otherwise specified.

### EXAMPLES

### Example 1

This examples illustrates improvements in lifespan and body weight in response to a triple therapy of the present teachings.

Lifespan was recorded as the age of the animal on the date of death or sacrifice. Animals were sacrificed when they became moribund as defined as one or more of the following: losing more than 25% of their maximal body weight, ataxia severe enough to impair ability to eat or drink, or lack of response to tactile stimulus. Unless otherwise noted, all animals were sacrificed by anesthetic overdose and perfused transcardially with phosphate buffered saline until the liver was cleared of blood.

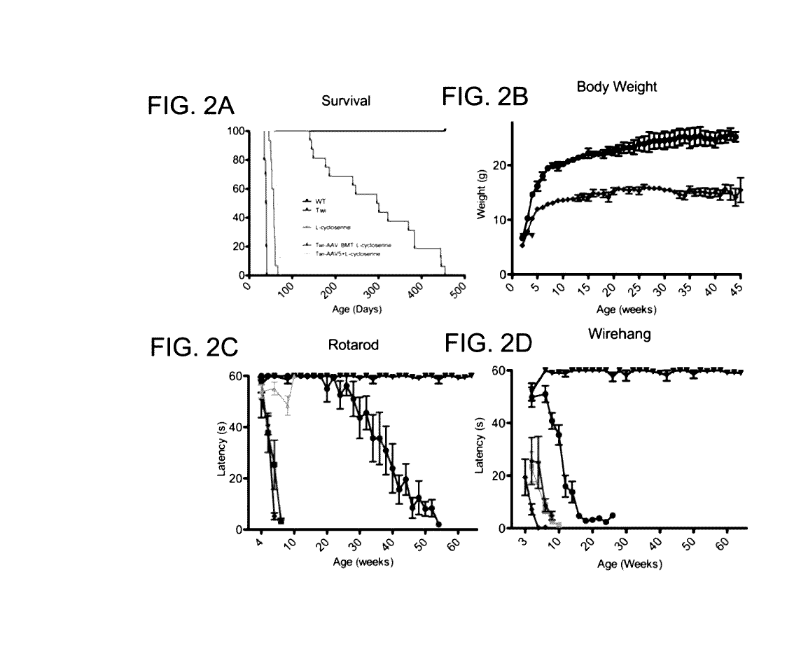

Untreated Twitcher animals had a median lifespan of 39.5 days. FIG. 2A depicts Kaplan-Meier curves comparing survival of Twitcher mice treated with AAV, BMT+L-cycloserine (“triple-treatment”) (n=16), AAV+L-cycloserine (n=10), or L-cycloserine alone (n=9) to untreated Twitcher mice (n=9) and untreated WT mice (n=10). The median lifespan of triple-treated Twitcher mice (298.5 d, range 160 d-454 d) was significantly greater than that of untreated Twitcher mice (39.5 d). Triple-treated Twitcher mice also lived significantly longer than Twitcher mice treated with AAV+L-cycloserine (70 d) or L-cycloserine alone (57 d, p<0.01).

The inventors previously showed that BMT or CNS-directed AAV-mediated gene therapy initiated in newborn Twitcher mice increased the median life span to ˜45 and ˜70 days, respectively (Lin et al., 2007 Molecular Therapy 15:44-52.; Reddy et al., 2011 J Neurosci 31:9945-9957). In the current study, the median lifespan for mice treated with L-cycloserine alone or L-cycloserine plus CNS-directed gene therapy is ˜57 and ˜70 days, respectively. In contrast, triple-treated Twitcher mice had a median lifespan of 298.5 days, with a range of 160-454 days. Additionally, several of the treated male mice were able to impregnate normal females, whereas untreated Twitcher males are not able to breed. Treated females were housed exclusively with other females.

Bodyweight was recorded for each mouse (n=12) once a week. Twitcher mice have impaired weight gain, with weight at death approximately half that of age-matched GALC +/+ littermates (an average of 8 g vs 16 g). FIG. 2B illustrates that the average body weight of triple-treated Twitcher mice was significantly greater than that of untreated Twitcher mice at 36 d. Triple-treated mice have increased weight gain compared to untreated Twitcher mice, though not to the same extent as WT animals (maximum weight ˜18 g vs 25 g for a WT mouse). The weight of triple-treated animals was stable until immediately preceding death.

### Example 2

This example illustrates improvements in neurological motor function in response to triple treatment.

Beginning at week 3, mice were tested every other week for performance on the rotarod and inverted wire-hang tests as previously described (Reddy et al., 2011 J Neurosci 31:9945-9957). The maximum length of either test was 60 s. Each animal was given at least 10 minutes to recover between trials or tests. Rotarod: Mice were gently placed on a stationary rod, then the rotation started. The rod was set to rotate at a constant 3 RPM. Latency to fall was recorded. The average of three trials was recorded. Wirehang: Animals were placed upright on a rigid ¼-inch wire mesh screen. The screen was gently shaken to encourage gripping then turned upside down 12 inches over soft bedding. Latency to hill was recorded.

Twitcher mice have impaired motor function as assessed through the rotarod and inverted wire-hang tests. Untreated Twitcher mice are unable to perform a test of coordination (rotarod) by 3-4 weeks of age. On average, triple-treated mice can perform the rotarod test normally until approximately 30 weeks of age, then slowly decline in their ability (FIG. 2C). Although the mean latency to fall steadily decreases over time, individual triple-treated mice typically perform normally (60 sec) until 1.5-2 months before they die. For example, two triple-treated Twitcher mice that lived to 450 and 454 days of age were able to stay on the rotarod for 60 sec until 400 and 395 days, respectively. The inverted wire-hang is a test of grip strength and is used as a surrogate for peripheral nerve function. Average latency to fall on the wire-hang test of motor function was recorded every two weeks.

Untreated Twitcher mice are unable to hang onto an inverted wire screen for the full 60-second test even at an early stage of disease. Triple-treated animals can perform this task better than untreated mice, but still show significant deficits compared to WT animals (FIG. 2D).

### Example 3

This example illustrates whole-brain GALC activity in triple-treated Twitcher mice.

In these experiments, following perfusion to sacrifice the animals (see Example 1), one brain hemisphere was homogenized in ddH2O. The homogenate was centrifuged and the supernatant collected. GALC activity was measured as previously described using a 3H-galactosylceramide (Lin et al, Molecular Therapy 12:422-430, 2005). After the reaction, uncleaved substrate was removed through galactose-saturated chloroform/methanol extraction. The remaining radioactivity, in the form of free ³H-galactose, was measured in a scintillation counter. The specific activity of GALC enzyme was calculated as nmols of substrate cleaved per hour per mg of protein.

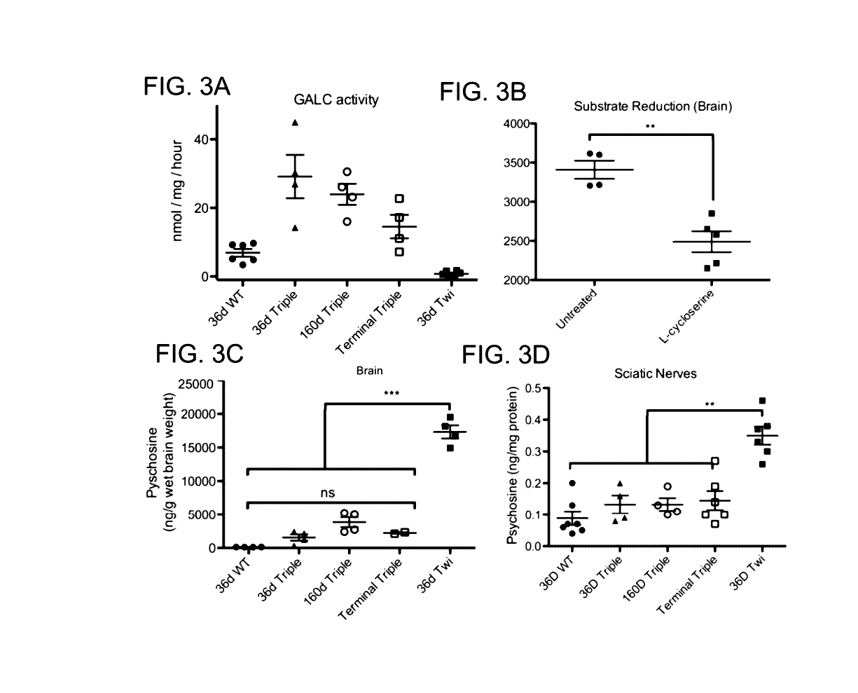

Twitcher mice have virtually undetectable whole-brain GALC activity as measured by cleavage of the radio-labeled natural substrate of GALC, galactosylceramide (FIG. 3A). At 36 days, the level of whole-brain GALC activity in triple-treated Twitcher mice is ˜3.5 fold greater than WT animals (4-6 animals per group). Although there is an apparent decrease in GALC activity as the animals age, this is not statistically significant. Even at a terminal time point, triple-treated Twitcher mice still have nearly double the WT levels of brain GALC activity.

### Example 4

This example illustrates the amelioration of psychosine accumulation in triple treated mice.

In these experiments, psychosine was measured as previously described. Briefly, one brain hemisphere or two sciatic nerves from a single mouse were homogenized in 0.04M citric acid. 50 μL of each sample was added to 20 μL of N,N-dimethylpsychosine (250 ng/mL) internal standard and 200 μL. MeOH. Samples were vortexed and centrifuged, then the supernatant collected. This extraction was repeated on the remaining pellet and the supernatants pooled. Psychosine concentrations were obtained using a column-switching LC-MS/MS method. Detection was achieved using an AB SCIEX 4000QTRAP tandem mass spectrometer (Applied Biosystems/MDS Sciex Inc., Ontario, Canada) employing ESI in the positive ion mode along with multiple reaction monitoring (MRM). Analyst software (version 1.5.1, Applied Biosystems/MDS Sciex Inc., Ontario, Canada) was used for the data analysis. The calibration curves (analyte peak area/internal standard peak area for Y-axis and analyte concentration for X-axis) of psychosine were obtained using the least square linear regression fit (y=ax+b) and a weighting factor of 1/x². The coefficient of determination (r²) was set as >0.98 for acceptance criteria of calibration curves.

Whole-brain psychosine levels are elevated in Twitcher mice relative to WT mice (FIG. 3B, FIG. 3C). Thrice-weekly injections of L-cycloserine alone significantly reduces whole-brain psychosine (FIG. 3B). Previous studies have shown that AAV2/5-mediated gene therapy alone reduces whole-brain psychosine levels, whereas BMT alone does not (Reddy et al., 2011 J Neurosci 31:9945-9957). Triple-treated Twitcher mice also show reduced psychosine levels in the brain as compared to untreated Twitcher mice (FIG. 3C). Triple therapy significantly reduced whole-brain psychosine levels in Twitcher mice at 36 d. Psychosine levels remained low at terminal time points (n=4-5 animals per group). Although whole brain psychosine levels in triple-treated animals are likely higher than WT, this difference is not statistically significant. Whole-brain psychosine levels remain low throughout the lifespan of triple-treated mice, with levels indistinguishable from WT at 160 d and at terminal time points. Psychosine also accumulates in the sciatic nerve of Twitcher mice. This accumulation is not observed in triple-treated Twitcher mice, even at terminal time points (FIG. 3D). Sciatic nerve psychosine levels in Twitcher mice were significantly reduced by triple therapy at every time point (n=4-7 animals per group). (**=P<0.01, ***=P<0.001).

### Example 5

This example illustrates the effect of triple treatment on BMT efficacy.

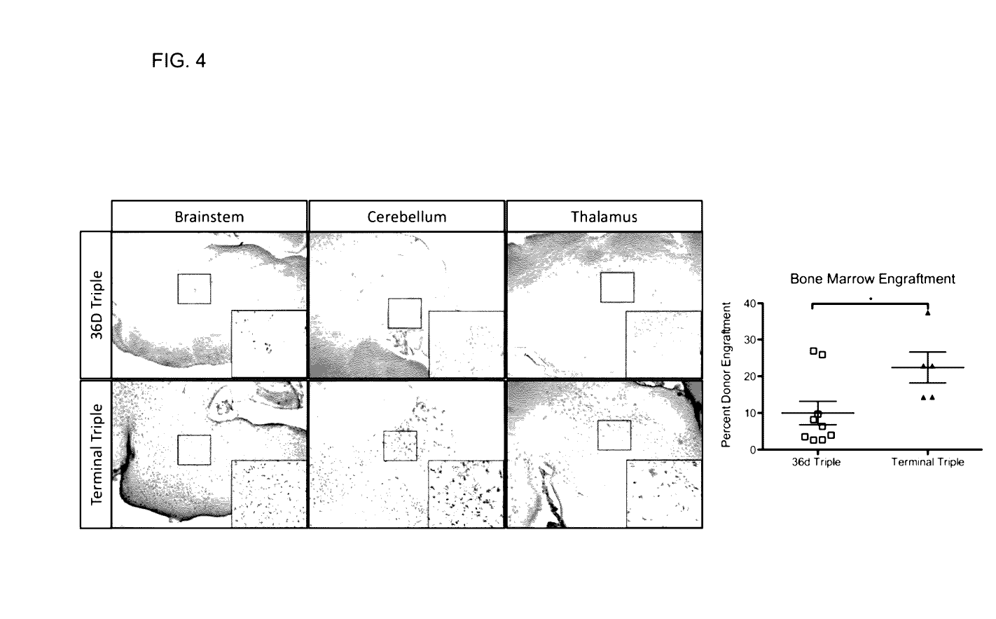

By 36 d, triple-treated animals have an average of 9.9% (range=5-30%) donor chimerism in the bone marrow (FIG. 4, Bone Marrow Engrafiment). There is a significant increase in average donor bone marrow engraftment (22.4%) in terminal triple-treated mice. Engraftment of donor microglia in the brain is a much slower process (Kennedy and Abkowitz, 1997 Blood 90:986-993). At 36 d, only rare GFP-positive cells are observed in the brainstem, cerebellum, and thalamus of triple-treated mice (FIG. 4, 36D Triple). In contrast, numerous GFP-positive cells can be seen in the brains of terminal animals, particularly in the regions of the brain stem, cerebellum and thalamus (FIG. 4 Terminal Triple). There is a significant increase in donor bone marrow engraftment (Bone Marrow Engraftment) in terminal triple-treated Twitcher mice (22.4%) compared to 36 d triple-treated Twitcher mice (9.9%) as determined by flow cytometry. (*=p<0.05) While triple-treated Twitcher mice have very few donor cells in the brain at 36 days of age (34 days post-transplant), the number of donor-derived (GFP-positive) cells increased in the brains of animals that were >160 days of age.

### Example 6

This example illustrates changes in myelination and ‘globoid cell’ infiltration resulting from triple treatment of Twitcher mice.

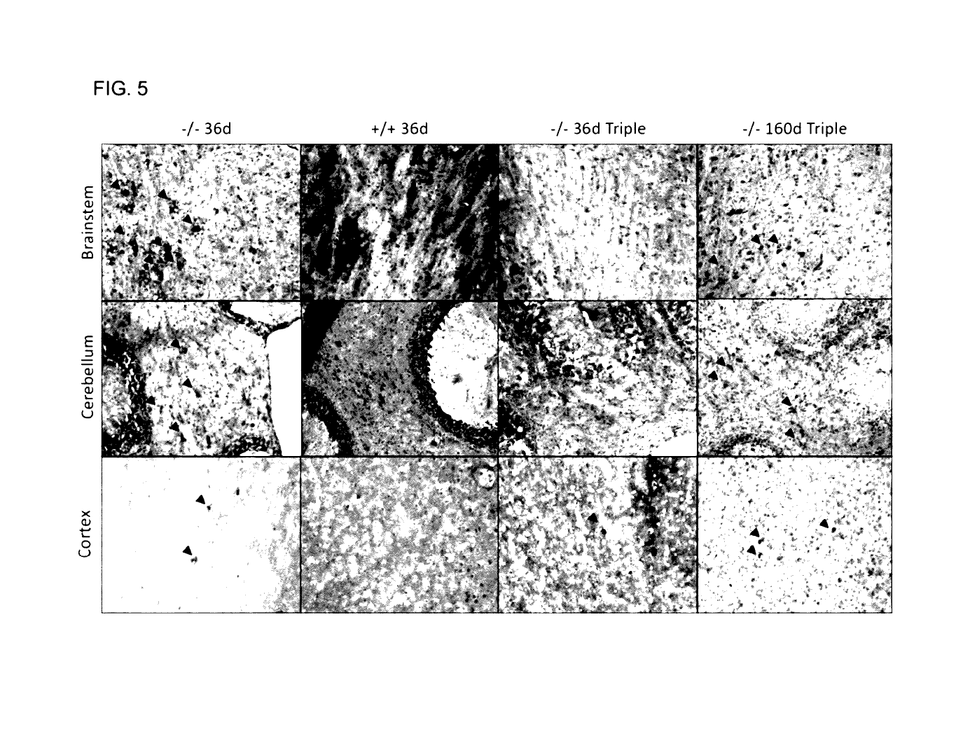

In these experiments, Luxol fast blue/periodic acid Schiff (LFB/PSAS) staining was performed to assess demyelination in the cortical, brain stem and cerebellar white matter. FIG. 5 shows representative staining in each of these brain tissues. No substantial myelin loss was seen in 36 d triple-treated mice (−/−36 d triple) compared to age-matched normal controls (+/+36 d). Older triple-treated mice (−/−160 d triple) show multifocal demyelination of all three evaluated areas. The degree of demyelination is similar to that observed in the untreated mice (−/−36 d). Brain stem white matter tracts, followed by subcortical white matter, demonstrated more severe demyelination. In addition, a considerable decrease in PAS-positive globoid cells (arrow heads), from total absence to very rare, was seen in 36 d triple-treated mice (−/−36 d triple) when compared to untreated mice (−/−36 d). Older triple-treated mice (−/−160 d triple) showed a considerable number of PAS-positive globoid cells. However, they appeared smaller and with less PAS-positive material, when compared to untreated mice (−/−36 d) (original magnification 200×).

There is a substantial decrease in LFB staining in the cortex, brain stem and cerebellum of untreated 36 day old Twitcher mice compared to age-matched normal controls (FIG. 5). There is also an increase in the number of PAS-positive ‘globoid cells’ in the untreated 36 day old Twitcher mice compared to the normal controls. The level of LFB staining in 36 day old triple-treated Twitcher mice is comparable to the normal control. Although there are still some ‘globoid cells’ present in the 36 day triple-treated Twitcher mice the number of ‘globoid cells’ is greatly reduced compared to age-matched Twitcher mice. The intensity of LFB staining in 160 day triple-treated Twitcher mice is comparable to the 36 day untreated Twitcher mice. Although the number of PAS-positive cells in 160 day triple-treated Twitcher mice is comparable to 36 day untreated Twitcher mice, the cells appear smaller with less PAS-positive material.

### Example 7

This example illustrates the effect of triple treatment on microglial and astrocyte activation.

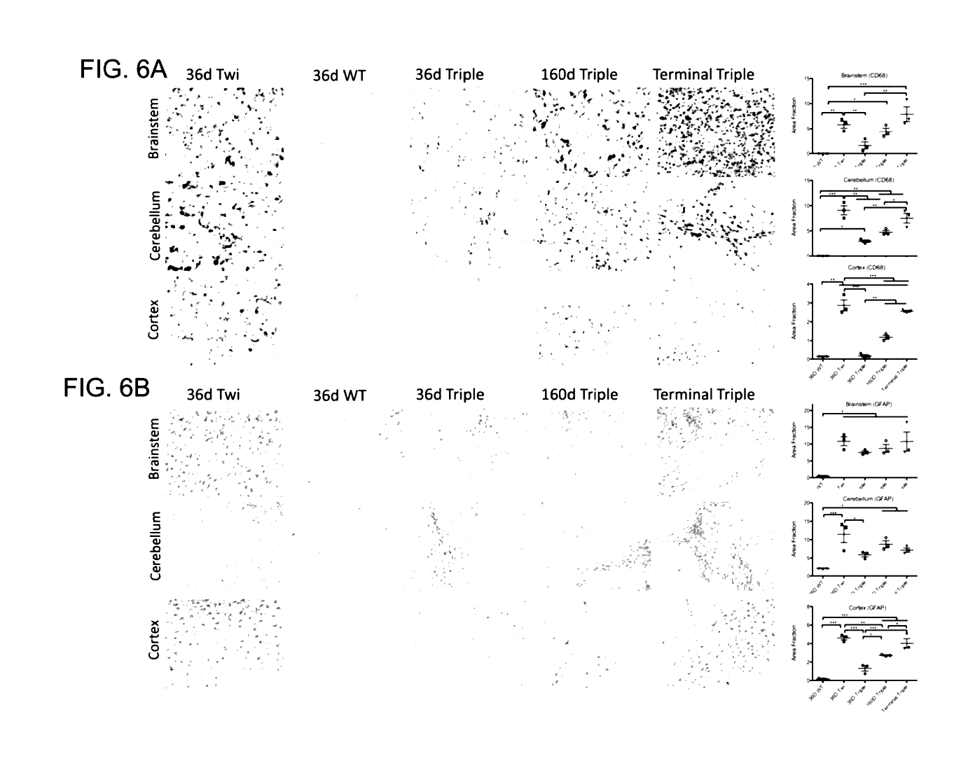

As previously reported, an increased number of CD68-positive macrophages and activated microglia are found in the brains of Twitcher mice compared to WT controls at 36 d (Lin et al., 2007 Molecular Therapy 15:44-52.; Reddy et al., 2011 J Neurosci 31:9945-9957). Untreated Twitcher animals show large populations of CD68-positive cells relative to WT in all brain regions examined. Decreased staining is observed in 36 d triple-treated Twitcher brains, though levels appear to increase at 160 d and at terminal time points. Triple treatment dramatically decreased CD68 staining at 36 d (FIG. 6A). However, CD68 staining steadily increases as the treated mice age. A similar trend is observed for GFAP-positive activated astrocytes (FIG. 61). Average area fraction positive for CD68 was quantified (n=3 animals per group). CD68 staining was significantly reduced in triple-treated Twitcher brains relative to untreated Twitcher brains at 36 d. CD68 staining increased as triple-treated animals aged. A similar pattern of GFAP staining was observed for untreated and triple-treated mice. Average area fraction positive for GFAP was significantly reduced in Twitcher brains by triple therapy at 36 d, but showed a significant increase as animals aged. (*=p<0.05, **=P<0.01, ***=P<0.001). Astrocytosis is diminished in triple-treated Twitcher brains at 36 d, but increases as the animals age.

### Example 8

This example illustrates the effect of triple treatment on cytokine and chemokine levels.

To further characterize the effect of triple therapy on neuroinflammation in the Twitcher brain, cytokine/chemokine levels were measured in whole-brain homogenates.

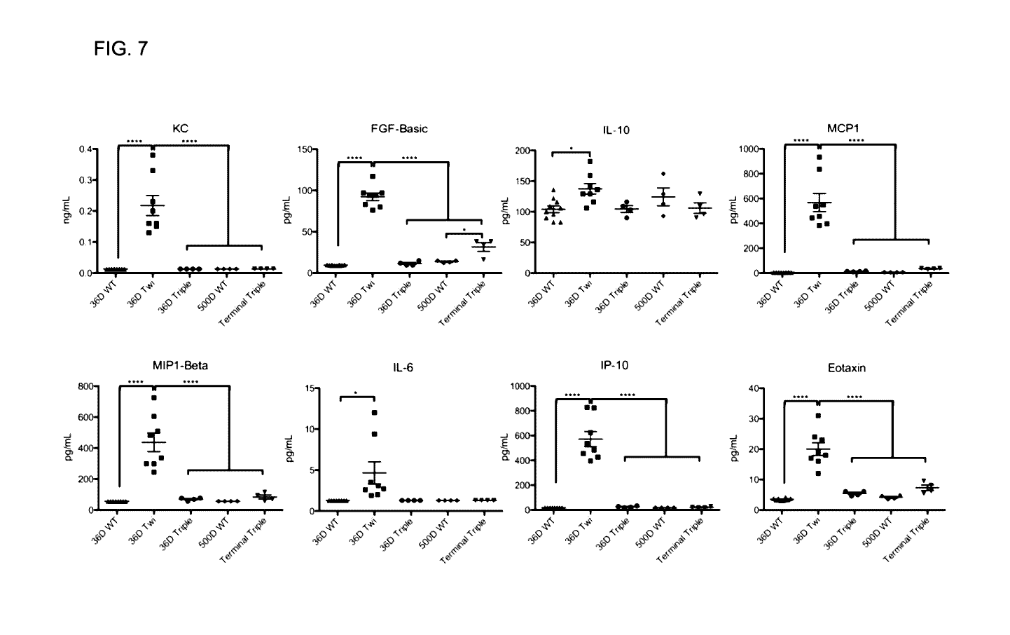

Several pro-inflammatory cytokines and chemokines were increased in Twitcher brains relative to WT at 36 d (FIG. 7). Expression of these immune mediators was reduced to WT levels in 36 d triple-treated mice. Inflammatory cytokines and chemokines (KC, FGF-Basic, MCPI, MIP-β, IL-6, IP-10, Eotaxin) were increased in untreated Twitcher brains relative to WT at 36 d (n=4-10 animals per group). Triple therapy resulted in a significant decrease in pro-inflammatory cytokines and chemokines. The levels remained indistinguishable from WT at terminal time points, with the exception of a slight increase in FGF-basic. IL-10, a cytokine with anti-inflammatory functions, was significantly increased in untreated Twitcher brains relative to WT. Triple-treated Twitcher brains showed IL-10 levels that were indistinguishable from WT at 36 d and at terminal time points. (*=p<0.05, ****=P<0.0001) The majority of these cytokines remained low throughout the lifespan of the treated animals, with only FGF-basic showing a slight increase in terminal mice. Interestingly, IL-10, a cytokine with immune suppressive effects, was minimally elevated in untreated Twitcher brains, and was reduced to WT levels following triple therapy.

All cited references are incorporated by reference, each in its entirety. Applicant reserves the right to challenge any conclusions presented by the authors of any reference.

1. A method of treating a lysosomal storage disease in a subject in need thereof, comprising:
administering a therapeutically effective amount of a vector encoding galactocerebrosidase (GALC);
administering a substrate reduction therapy; and
administering an immunomodulation therapy.

2. A method of treating a lysosomal storage disease in accordance with claim 1, wherein the lysosomal storage disease is globoid-cell leukodystrophy (GLD).

3. A method of treating a lysosomal storage disease in accordance with claim 1, wherein the vector encoding galactocerebrosidase (GALC) is an adeno-associated virus (AAV).

4. A method of treating a lysosomal storage disease in accordance with claim 3, wherein the AAV is selected from the group consisting of AAV2/5, AAVrh10, and AAV2/9.

5. A method of treating a lysosomal storage disease in accordance with claim 1, wherein the administering a substrate reduction therapy comprises administering an inhibitor of an enzyme that acts immediately upstream of galactosylceramide and galactosylsphingosine (psychosine).

6. A method of treating a lysosomal storage disease in accordance with claim 6, wherein the enzyme that acts immediately upstream of galactosylceramide and galactosylsphingosine (psychosine) is ceramide galactosyltransferase (CGT).

7. A method of treating a lysosomal storage disease in accordance with claim 1, wherein the administering a substrate reduction therapy comprises administering a therapeutically effective amount of an inhibitor of an enzyme that participates in ceramide synthesis.

8. A method of treating a lysosomal storage disease in accordance with claim 7, wherein the inhibitor of an enzyme that participates in ceramide synthesis is an inhibitor of 3-ketodyhydrosphingosine synthase.

9. A method of treating a lysosomal storage disease in accordance with claim 8, wherein the inhibitor of 3-ketodyhydrosphingosine synthase is L-cycloserine.

10. A method of treating a lysosomal storage disease in accordance with claim 1, wherein the immunomodulation therapy comprises bone marrow transplantation.

11. A method of treating a lysosomal storage disease in accordance with claim 1, wherein the administering an immunomodulation therapy comprises administering a therapeutically effective amount of a JAK-STAT inhibitor.

12. A method of treating a lysosomal storage disease in accordance with claim 6, wherein the JAK-STAT inhibitor is selected from the group consisting of 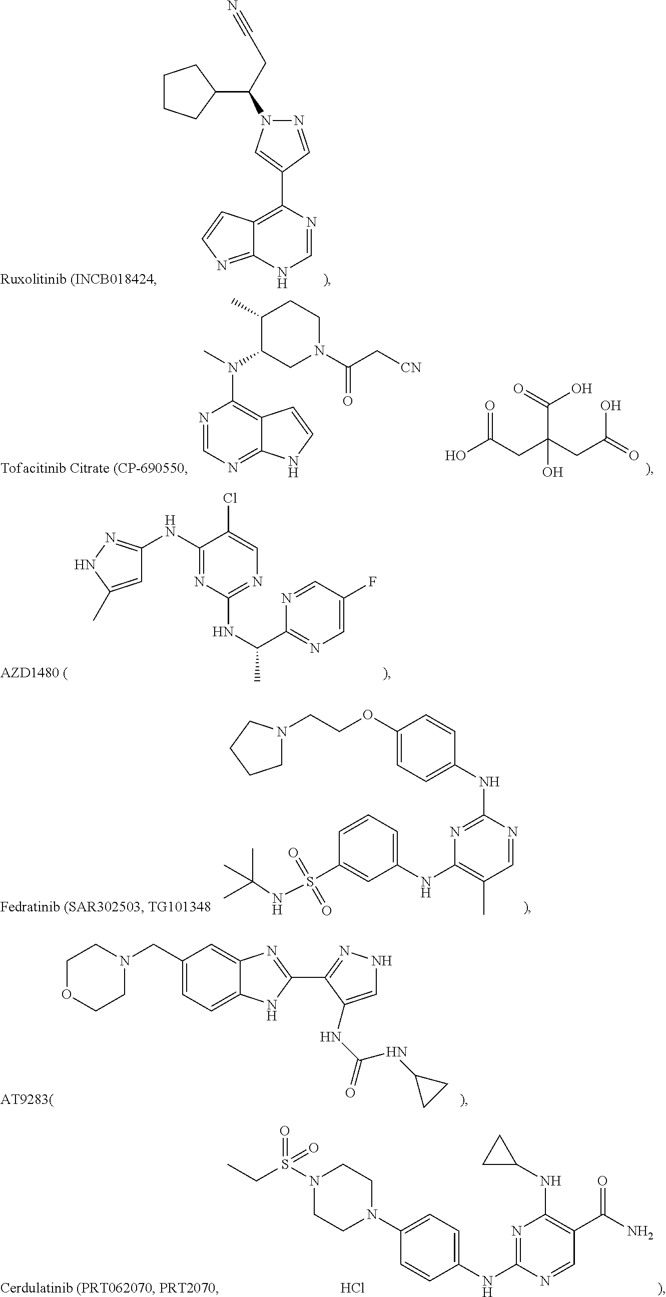 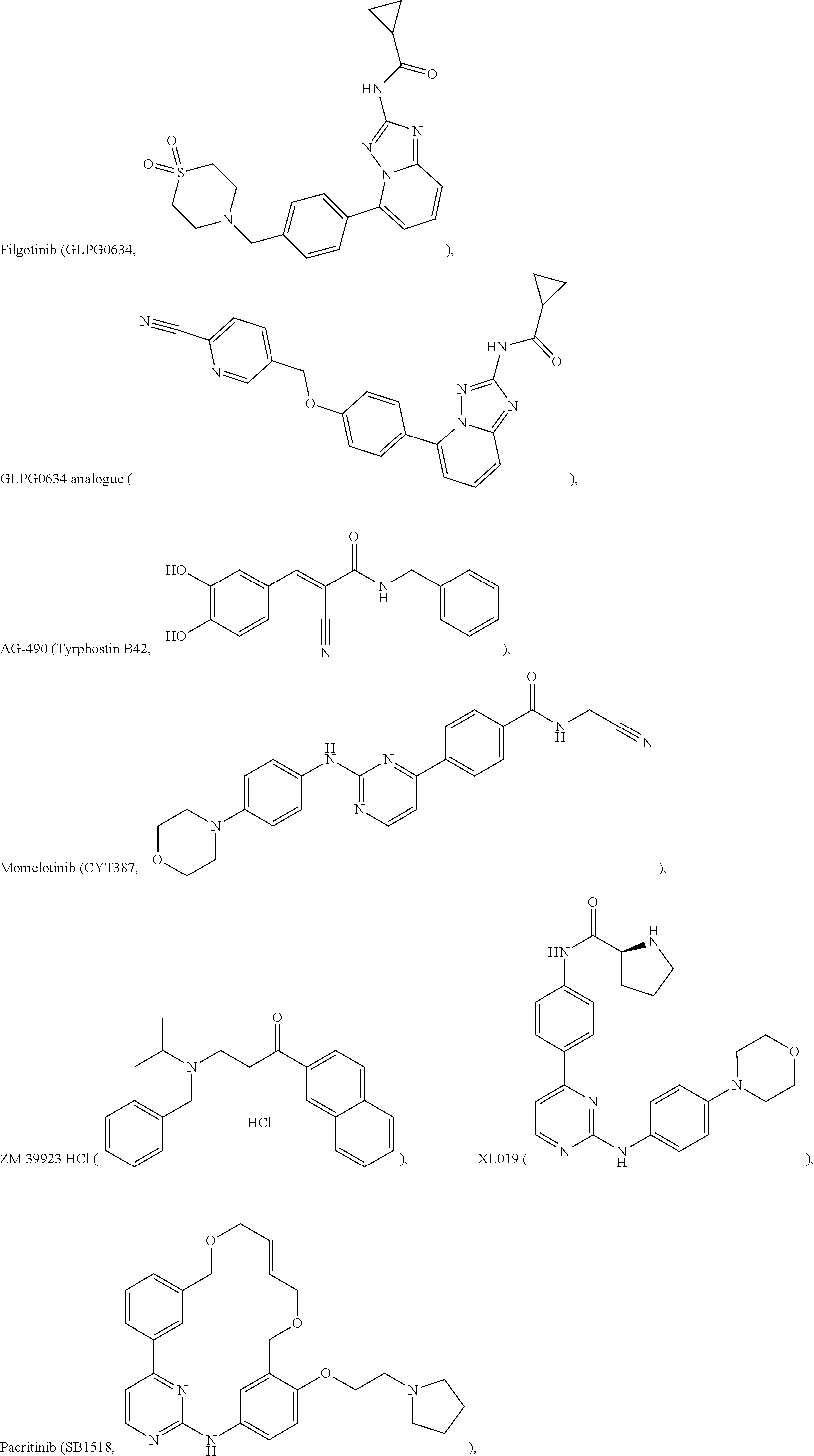 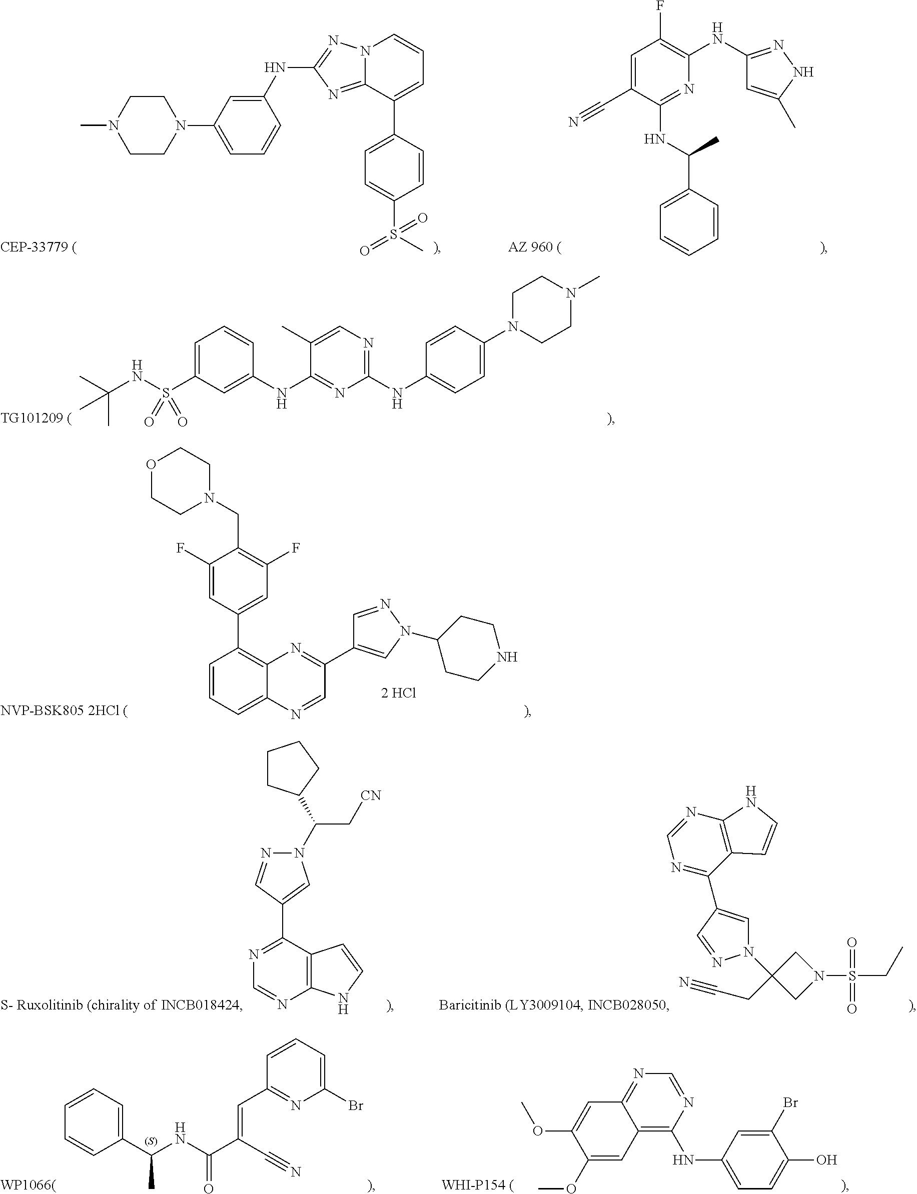 [chemical structure]
